(19) United States
(12) Patent Application Publication
(10) Pub. No.: US 2004/0199208 A1
(43) Pub. Date: Oct. 7, 2004

(54) High performance suture

(75) Inventor: Seth A. Foerster, San Clemente, CA (US)

Correspondence Address: Donald E. Stout, Stout, Uxa, Buyan & Mullins, LLP, Suite 300, 4 Venture, Irvine, CA, 92618, US

(73) Assignee: Opus Medical, Inc., San Juan Capistrano, CA (US)

(21) Appl. No.: 10/803,471

(22) Filed: Mar. 18, 2004

Related U.S. Application Data
(60) Provisional application No. 60/455,819, filed on Mar. 18, 2003.

(51) Int. Cl. A61L 17/00; A61B 17/04

(52) U.S. Cl. 606/228

(57) ABSTRACT

A high strength abrasion resistant surgical suture material with industry standard knot tying characteristics and color marking characteristics includes a core formed of a plurality of twisted fibers of a first material, surrounded by a braided cover including a first material sheathed in or coated by a second material different than the first material. The first material is preferably ultrahigh molecular weight polyethylene and the second material is preferably a polymeric material having good knot-tying characteristics.

This application claims the benefit of U.S. provisional patent application No. 60/455,819, filed on Mar. 18, 2003, the entire disclosure of which is incorporated herein by this specific reference.

### BACKGROUND OF THE INVENTION

The present invention relates to high strength surgical suture materials, and more particularly to braided suture blends of ultrahigh molecular weight polyethylene and polyester. The composite sutures have high tensile strength as well as excellent knot tying characteristics.

### DESCRIPTION OF THE RELATED ART

The strength of a suture, particularly the tensile strength of the suture, is an important consideration for any surgical suture material. One of the strongest materials currently formed into elongated strands is an ultrahigh molecular weight long chain polyethylene (UHMWPE), typically used for fishing line and the like, which is sold under the trade names such as Dyneema® or Spectra®. However, this material, while much stronger than ordinary surgical suture, does not have acceptable knot tying characteristics for use in surgical applications because of its low frictional coefficient. Additionally, this material only comes in one color, making multiple suture distinction difficult, especially arthroscopically.

Current braided suture technology allows for acceptable knot tying characteristics through use of the material properties afforded by silicone-coated polyester. Polyester also comes in a variety of colors making it easy for the manufacturer to assemble many color patterns and color schemes to assist the surgeon in sorting out the many sutures used in a procedure.

Suture knot holding characteristics are a function of the suture's ability to frictionally lock to itself within the knot. The magnitude of this friction is determined by the coefficient of friction of the material used to form the suture, as well as the geometry of the suture-to-suture interface within the knot. The relatively high coefficient of friction for polyester has made it the material of choice within the industry, while the standard interface in the industry is woven braid upon woven braid. The individual elements of the braid in this crossed up interplay leave small round elements laying across each other, producing point contacts between the elements. Point contacts result in relatively low friction, and thus result in less than optimum knot tying abilities.

Accordingly, there exists a need for improved suture materials having high tensile strength and excellent knot tying characteristics.

### SUMMARY OF THE INVENTION

The present invention advantageously provides a suture strand having high tensile strength as well as clinically acceptable knot-tying characteristics. Briefly, the suture strand comprises a core formed of a plurality of fibers of a first material, surrounded by a cover including a first material sheathed in or coated by a second material different than the first material. Preferably, the first material is a high strength, high tenacity material such as ultrahigh molecular weight long chain polyethylene, and the second material is a material having good knot-tying characteristics, such as a polymer selected, for instance, from the group consisting of PET, polyester, coated urethanes, and mixtures thereof.

In one embodiment of the invention, the core comprises a twisted strand of fibers of the first material, and the cover is braided around the core. Each fiber of the cover is individually sheathed in the second material. Alternatively, each fiber may be coated with the second material.

In another embodiment of the invention, the cover comprises a plurality of braided fibers, with a single, unitary sheath around the braid. Alternatively, the braid may be coated with the second material.

In any of the aforementioned embodiments, the cover may also include a secondary coating formed of a friction-enhancing material such as silicone. The cover may also be colored as desired to assist the surgeon or technician in sorting out and detecting the suture during a procedure.

More particularly, there is provided in one aspect of the invention a suture strand suitable for use as a suture or ligature, which comprises a core formed of a plurality of fibers of a first material, and a cover surrounding the core, wherein the cover includes the first material and a second material different than the first material.

In another aspect of the invention, there is provided a suture strand suitable for use as a suture or ligature, which comprises a first covering component formed of ultrahigh molecular weight polyethylene and a second covering component formed of a polymeric material surrounding the cover.

Additional aspects and advantages of the present invention are set forth in the following description and claims, particularly when considered in conjunction with the accompanying drawings in which like parts bear like reference numerals.

### BRIEF DESCRIPTION OF THE DRAWINGS

FIG. 1 is a cross-sectional view of a suture strand according to the present invention;

FIG. 2 is a cross-sectional view of a suture strand according to an alternate embodiment of the invention;

FIG. 3 is a perspective view, partially broken away, of the suture strand of FIG. 1;

FIG. 4 is a perspective view, partially broken away, of the suture strand of FIG. 2;

FIG. 5 is a cross-sectional view of a suture strand according to still another embodiment of the invention; and

FIG. 6 is a perspective view, partially broken away, of the suture strand of FIG. 5;

### DETAILED DESCRIPTION OF THE DRAWINGS

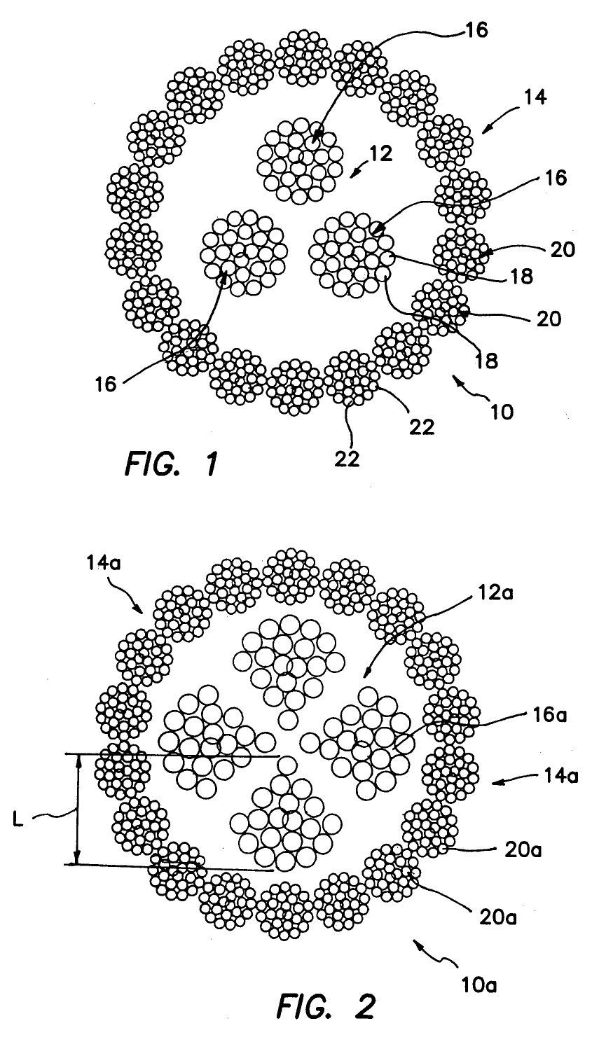

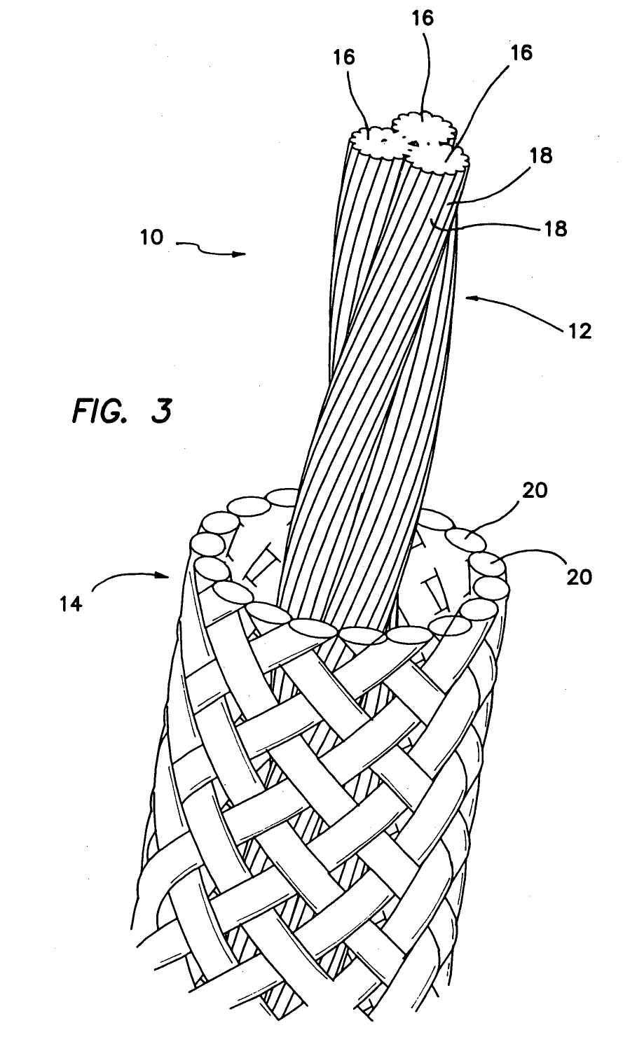

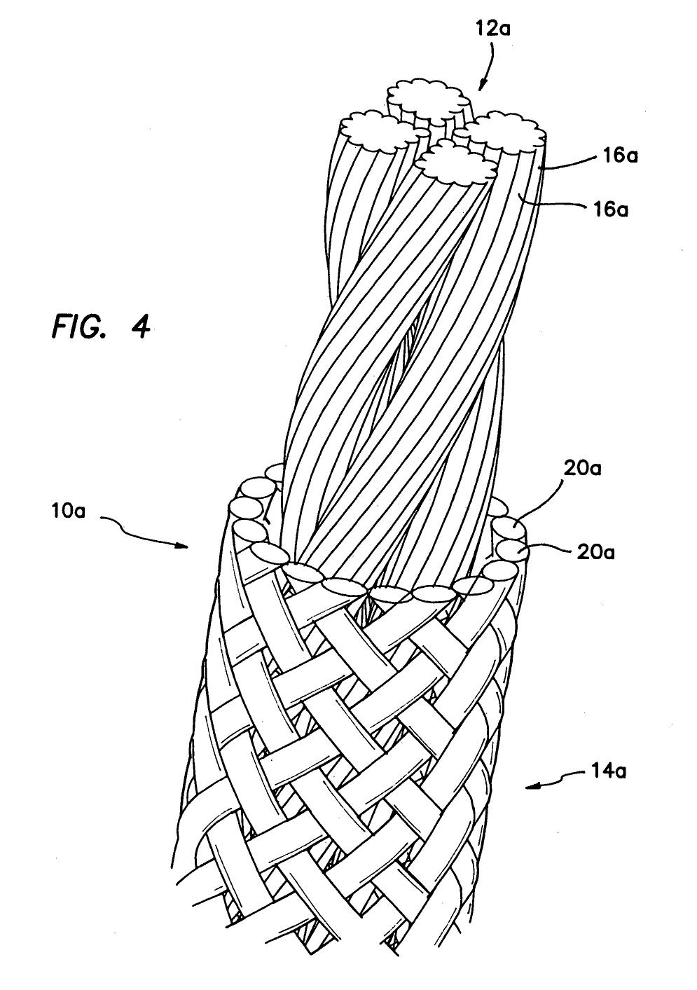

Referring now to FIGS. 1 and 3, a suture strand 10 according to the present invention comprises a core 12 and a cover 14. The core 12 comprises several, for example three, fibers 16 twisted together to form a single core element. Each fiber 16 itself comprises a bundle of filaments 18 of a first material. The cover 14 comprises an annular woven braid including a plurality of composite fibers 20 surrounding the core 12. The illustrated example comprises twelve such composite fibers 20, but any number could be employed. Each composite fiber 20 comprises a bundle 22 of filaments 24 of the same material as the filaments 18 in the core 12. Each bundle 22 is individually encased in a sheath or coating 26 of a second material different than the first material.

The first material, i.e. the material used to form the filaments 18 and 24, is preferably a high strength, high molecular weight, high tenacity material such as ultrahigh molecular weight long chain polyethylene. The second material, i.e. the material used to form the sheath or coating 26 of each composite fiber 20, is preferably a material having clinically acceptable knot tying characteristics. Preferably the second material is also opaque and available in a wide variety of colors. For example, the second material may be selected from the group consisting of PET, polyester, coated urethanes, and mixtures thereof.

FIGS. 2 and 4 show a suture strand 10′ according to an alternate embodiment of the invention. The strand 10′ comprises a core 12′ and a composite cover 14′. As in the previous embodiment, the core 12′ comprises a plurality of twisted fibers 16′ of the first material. The composite cover 14′ comprises an inner ring 28 formed of the first material and an outer sheath 30 formed of the second material. The inner ring 28 preferably comprises an annular woven braid including a plurality of, for instance twelve, fibers 20′, each fiber 20′ comprising a plurality of twisted filaments 22′ of the first material. The outer sheath 30 preferably fits tightly around the inner ring 28.

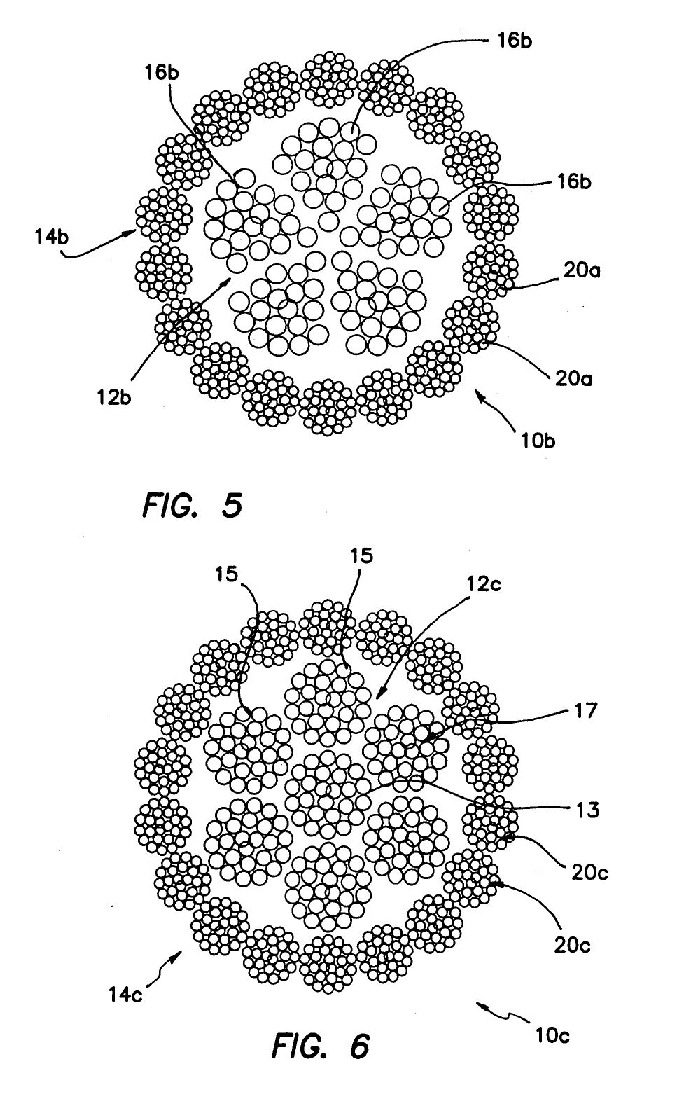

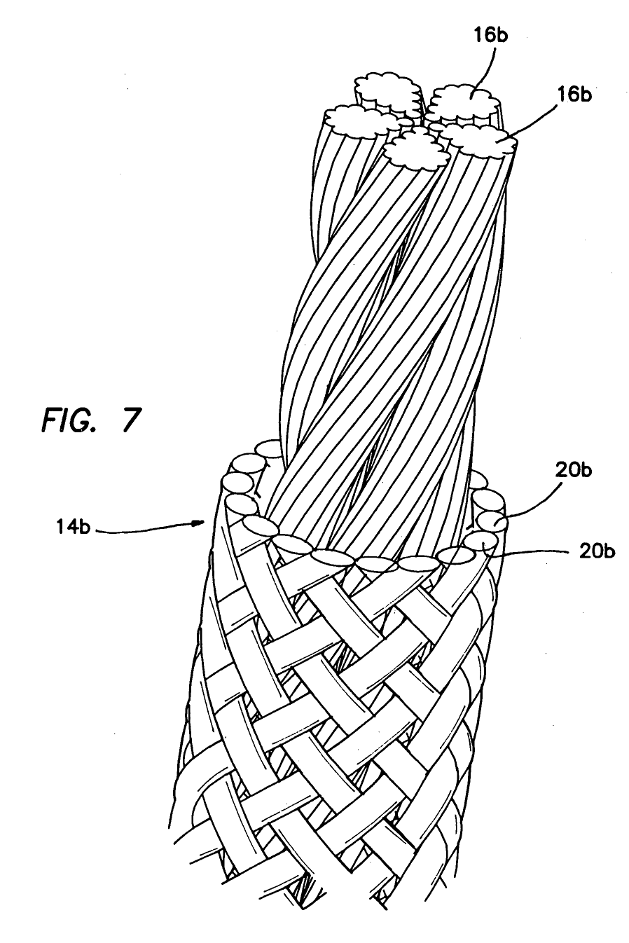

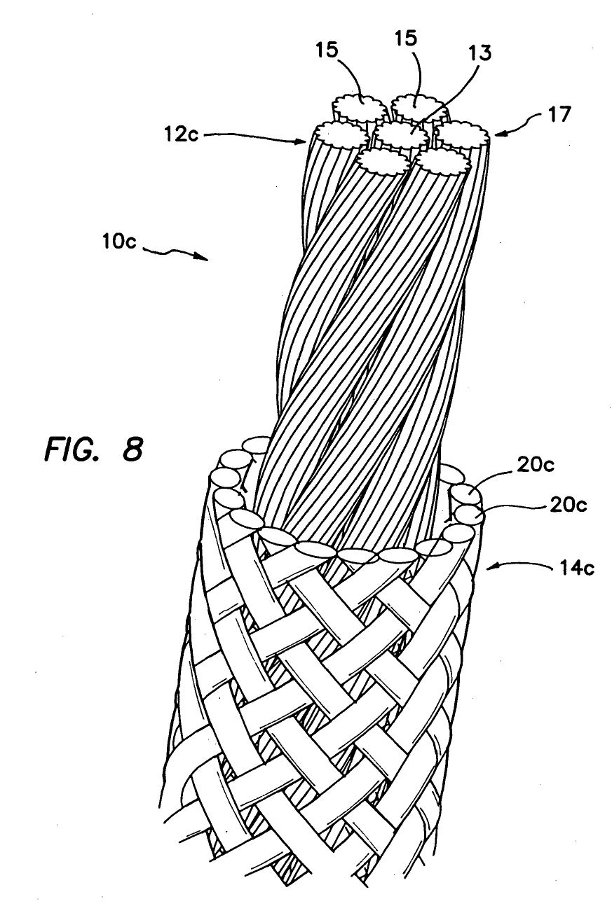

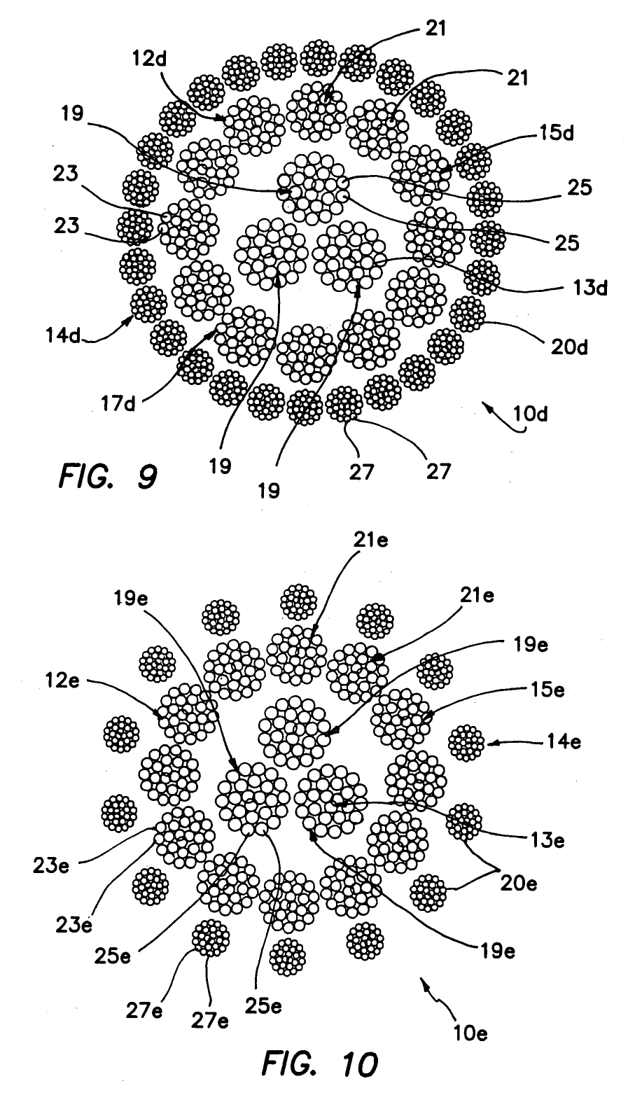

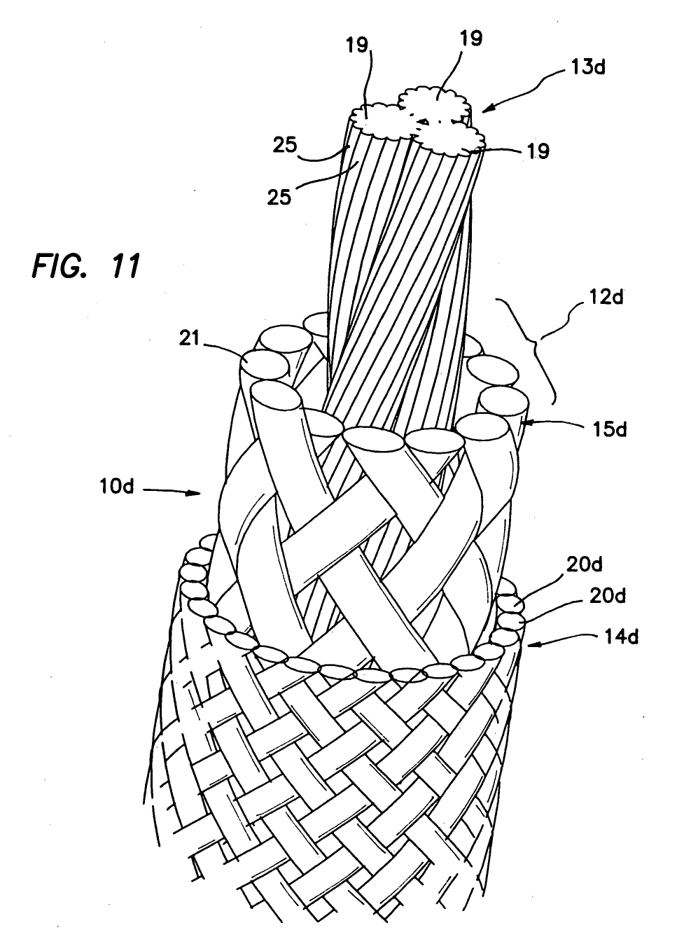

FIGS. 5 and 6 show a suture strand 10″ according to still another embodiment of the invention. As in the previous embodiment, the strand 10′ comprises a core 12″ and a composite cover 14″. The core 12″ is identical to the cores 12 and 12′ of FIGS. 1 and 2. Similarly, the inner ring 28″ is identical to the inner ring 28 of FIG. 2. The outer sheath of FIG. 2, however is replaced by a coating 30 formed of the second material. Unlike the sheath, which has a circular inner circumference, the coating 30 conforms closely to the braided outer periphery of the inner ring 28″, thus increasing the amount of surface area available for interface when tying knots.

The knot holding abilities of the suture 10″ may be improved still further by providing a secondary coating 32 of a friction-enhancing material such as silicone. Such a coating may optionally also be provided on sutures according to the embodiments of FIGS. 1 and 2.

Sutures according to the present invention have been found to possess an optimum blend of characteristics including high strength, good frictional interface, and availability in a wide range of colors. For instance, a suture having the strength of an Ethibond® #5 suture has combined with the diameter, feel and knot-tying ability of a #2 suture is attainable using the principles of the present invention. Accordingly, the suture of the present invention, which can be attached to a suture anchor or curved needle, is ideally suited for a wide variety of surgical procedures and in particular, most orthopedic procedures, including rotator cuff repair, Achilles tendon repair, patellar tendon repair, ACL/PCL reconstruction, hip and shoulder reconstruction procedures, and replacement of suture in anchors.

While this invention has been described with respect to various specific examples and embodiments, it is to be understood that the invention is not limited thereto and that it can be variously practiced within the scope of the following claims.

What is claimed is:

1. A suture strand suitable for use as a suture or ligature, comprising:
a core formed of a plurality of fibers of a first material;
a cover surrounding the core, the cover including the first material and a second material different than the first material.

2 A suture strand according to claim 1, wherein the first material comprises a high tensile-strength material.

3. A suture strand according to claim 1, wherein the first material is a high molecular weight, high tenacity material.

4. A suture strand according to claim 1, wherein the first material is ultrahigh molecular weight polyethylene.

5. A suture strand according to claim 1, wherein the second material is a polymer selected from the group consisting of PET, polyester, coated urethanes, and mixtures thereof.

6. A suture strand according to claim 1, wherein the second material is substantially opaque.

7. A suture strand according to claim 1, wherein the first material is substantially transparent.

8. A suture strand according to claim 1, wherein the core comprises a plurality of fibers of the first material twisted together to form a core element.

9. A suture strand according to claim 8, wherein each fiber comprises a bundle of filaments of the first material.

10. A suture strand according to claim 1, wherein the cover is braided around the core.

11. A suture strand according to claim 1, wherein the cover comprises a plurality of fibers of the first material, and wherein each fiber is individually sheathed in the second material.

12. A suture strand according to claim 1, wherein the cover comprises;
a plurality of fibers of the first material; and
a single, unitary sheath surrounding the entire cover.

13. A suture strand according to claim 1, wherein the cover comprises a plurality of fibers of the first material, and wherein each fiber is individually coated with the second material.

14. A suture strand according to claim 1, wherein the cover comprises:
a plurality of fibers of the first material; and
a coating surrounding the entire cover.

15. A suture strand according to claim 1, wherein the cover further comprises a secondary coating formed of a second material.

16. A suture strand according to claim 15, wherein the secondary coating comprises a friction-enhancing material.

17. A suture strand according to claim 15, wherein the secondary coating comprises silicone.

18. A suture strand suitable for use as a suture or ligature, comprising:
a first covering component formed of ultrahigh molecular weight polyethylene;
a second covering component formed of a polymeric material surrounding the cover.

19. A suture strand according to claim 18, wherein the first covering component comprises an annular braid formed of a plurality of fibers of ultrahigh molecular weight polyethylene.

20. A suture strand according to claim 19, wherein the second covering component comprises a plurality of sheath elements, each sheath element surrounding a different individual fiber of the first covering component.

21. A suture strand according to claim 18, wherein the second covering component is a single, unitary sheath surrounding the first covering component.

22. A suture according to claim 18, wherein the second covering component is a coating.

23. A suture according to claim 19, wherein the second covering component comprises a coating applied individually to each fiber of the first covering component.

24. A suture strand according to claim 18 wherein the polymeric material is selected from the group consisting of PET, polyester, coated urethanes, and mixtures thereof.

25. A suture strand according to claim 18, wherein the polymeric material comprises substantially opaque material.

26. A suture strand according to claim 18, wherein the first covering component surrounds a core of twisted fibers.

27. A suture according to claim 26, wherein the first covering component is braided around the core.

28. A suture according to claim 26, wherein the twisted fibers are formed of ultrahigh molecular weight polyethylene.

29. A suture according to claim 18, wherein the first covering component consists essentially of a plurality of fibers of ultrahigh molecular weight polyethylene.

30. A suture strand according to claim 18, wherein the second covering component further comprises a secondary coating formed of a material other than ultrahigh molecular weight polyethylene.

31. A suture strand according to claim 18, wherein the secondary coating comprises a friction-enhancing material.

32. A suture strand according to claim 18, wherein the secondary coating comprises silicone.

33. A suture strand according to claim 26, wherein:
the first covering component comprises an annular braid formed of a plurality of fibers, each of the fibers in the first covering component having a first diameter; and
each of the fibers in the core has a second diameter larger than the first diameter.
